(19) United States
(12) Patent Application Publication
(10) Pub. No.: US 2023/0051110 A1
(43) Pub. Date: Feb. 16, 2023

(54) MEDICAL STATION WITH MATERIAL COLLECTION POINTS AND CHANNELS

(71) Applicant: CareFusion 303, Inc., San Diego, CA (US)

(72) Inventors: Noe Arroyo, San Diego, CA (US); David Hoard, Escondido, CA (US)

(21) Appl. No.: 17/884,949

(22) Filed: Aug. 10, 2022

Related U.S. Application Data
(60) Provisional application No. 63/232,942, filed on Aug. 13, 2021.

(51) Int. Cl. G16H 20/10; G16H 10/60

(52) U.S. Cl. CPC G16H 20/10; G16H 10/60

(57) ABSTRACT

Features relating to a medical station are provided. The medical station includes collection points and channels for collection and diversion of materials. The medical station includes a base and an upper module, where the upper module is configured to cover a processing device and provide work areas for access and use of the processing device. The upper module, consistent with implementations of the current subject matter, includes recesses and channels for collecting spilled materials to prevent the spilled materials damaging critical components.

### CROSS REFERENCE TO PRIORITY APPLICATION

This application claims priority to U.S. Patent Application No. 63/232,942 filed on Aug. 13, 2021. The full disclosure is incorporated herein by reference in its entirety.

### TECHNICAL FIELD

The current subject matter described herein relates generally to a medical station. In particular, the current subject matter relates to a medical station with collection points and channels, receptacles, or any other structure for collection and diversion of materials.

### BACKGROUND

Medical stations may be incorporated in various clinical and healthcare environments. For example, a medical station may include a standing unit or base that incorporates a processing device such that a clinician or healthcare practitioner may operate and/or access the processing device at the standing unit or base. The processing device may be integral with or otherwise connected to the standing unit or base. The medical station may be incorporated with other stations and/or modules.

### SUMMARY

Aspects of the current subject matter relate to a medical station with collection points and channels to collect and divert materials away from sensitive areas of the medical station. In an example implementation, the medical station is a medical dispensing station or medication dispenser that is configured to dispense a medication.

In one aspect, there is disclosed a medical station, comprising: a base; and an upper module formed of a structure positioned atop the base, the upper module comprising a raised covering component and a planar support component. The raised covering component comprises a first top surface; a first recess formed within at least a portion of the first top surface; at least one channel extending from the first recess; and a plurality of sidewalls extending from respective edges of the first top surface. The planar support component comprises a second top surface extending from a distal end of a first sidewall of the plurality of sidewalls of the raised covering component, and a second recess formed within at least a portion of the second top surface.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a perspective view of an upper module of a medical station consistent with implementations of the current subject matter;

FIG. 2 is a side view of an upper module of a medical station consistent with implementations of the current subject matter;

FIG. 3 is a top view of an upper module of a medical station consistent with implementations of the current subject matter;

FIG. 4 is a back view of an upper module of a medical station consistent with implementations of the current subject matter;

FIG. 5 depicts aspects of a medication station consistent with implementations of the current subject matter; and

FIG. 6 depicts aspects of a medication station consistent with implementations of the current subject matter.

When practical, similar reference numbers denote similar structures, features, or elements.

### DETAILED DESCRIPTION

Aspects of the current subject matter relate to a medical station with collection points and channels to collect and divert materials away from sensitive areas (or other areas) of the medical station. The medical station may be a standalone module or a component of a modular system. The medical station may be part of a medication management system that includes various modules and stations for the secure storage and dispensing of medication.

Aspects of the current subject matter provide a medical station that has a base and an upper module, where the upper module connects to or is otherwise integral with the base. The upper module is a structure or frame positioned atop the base. In some implementations, the upper module may connect to more than one type of base. In some implementations, the base is a standing unit or module. The base may be used to store medication, supplies, and/or documentation such as medical records or files. In some implementations, the base may be a medication dispensing unit. In some implementations, the base may include a refrigeration and/or freezer unit.

The medical station in accordance with implementations of the current subject matter is provided with features including collection points, channels, receptacles, and/or other structures to safely and securely contain and direct materials that may be spilled on the medical station. For example, as the medical station may include electronic components, such as one or more processing devices, and other valuable or sensitive components, there is a need to protect these components from accidental spills of liquids or other materials that may occur. The medical station includes dedicated areas to catch the materials, and in some instances divert the materials, in such a way that the electronic components and other components are not damaged.

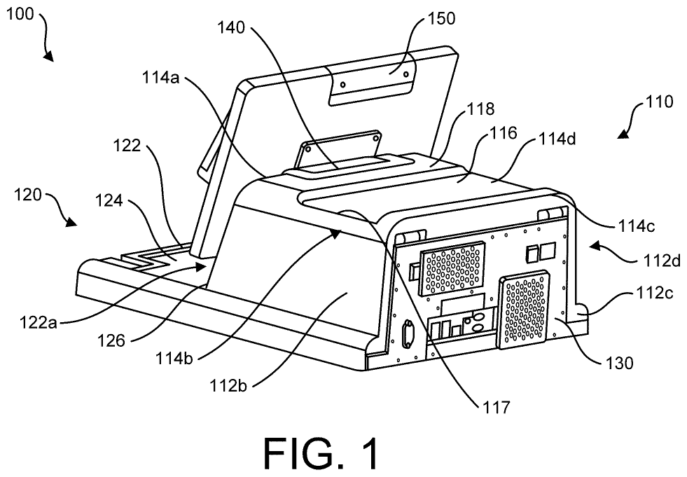

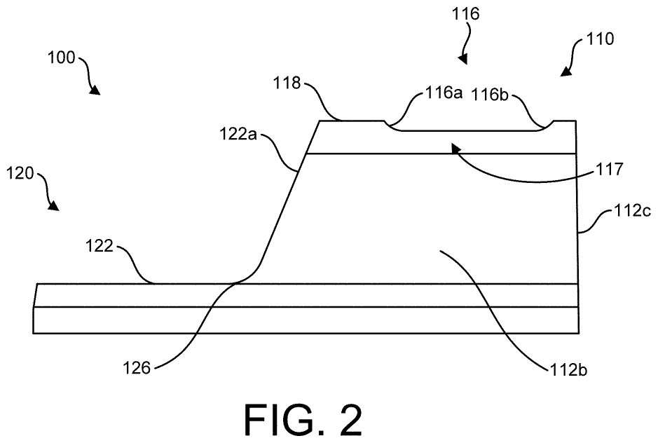

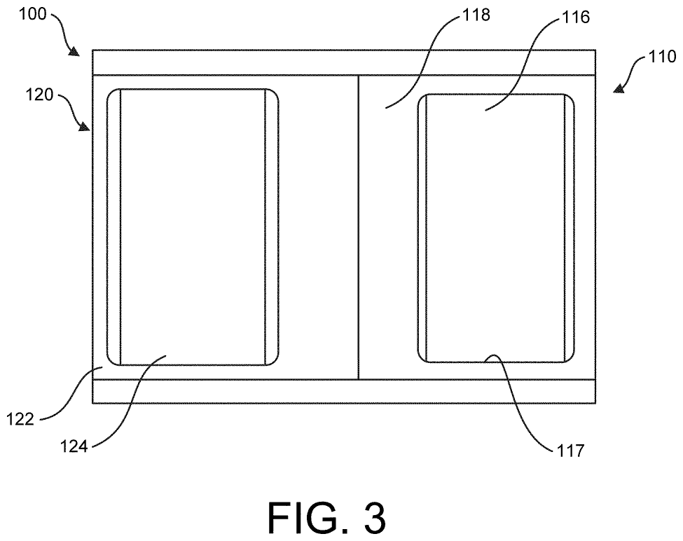

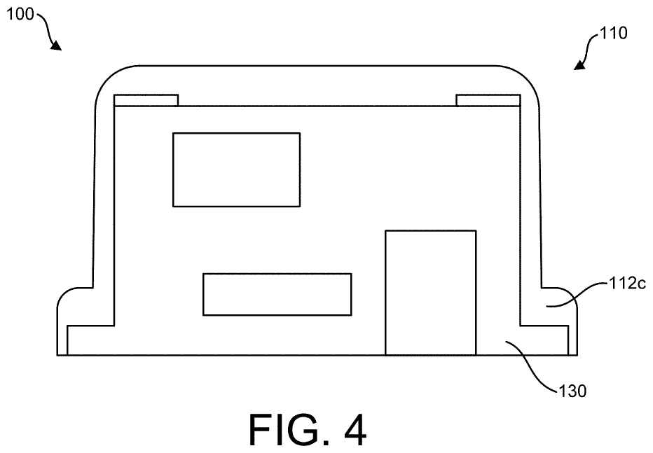

FIG. 1, FIG. 2, FIG. 3, and FIG. 4 depict various aspects of an upper module 100 of a medical station consistent with implementations of the current subject matter. FIG. 1 depicts a rear, perspective view of the upper module 100, FIG. 2 depicts a side view of the upper module 100, FIG. 3 depicts a top view of the upper module 100, and FIG. 4 depicts a rear view of the upper module 100.

The upper module 100 is shaped, configured and positioned on a base (not shown in FIG. 1-FIG. 4) to cover and protect a processing device and to provide a work surface for healthcare workers. The upper module 100, according to aspects of the current subject matter, includes a raised covering component 110 and a planar support component 120. The raised covering component 110 is shaped and positioned to cover and contain within a processing device. The planar support component 120 is shaped and positioned in front of the raised covering component 110 to provide a work surface for healthcare workers. A display unit 150 may couple to the upper module 100 to allow for the healthcare workers to access and use the processing device. The planar support component 120 has a recessed work surface to hold or contain items (such as medications, pens, papers, or other items.) In an implementation, the recessed work surface is sized and shaped to contain or capture a volume of fluid (such as to contain 1 liter of liquid) should a spill occur.

With reference to FIG. 1 and FIG. 2, the raised covering component 110 has a first top surface 118 and a first recess 116 formed within at least a portion of the first top surface 118. A channel 117 extends from the first recess 116. The channel is configured to divert liquid in the first recess 116 away from sensitive electronic components in the front and back, such as to the left and right side of the first recess 116. For example, the first recess 116 may have one or more edges or sides from which material contained in the first recess 116 may flow, allowing the material to be diverted from one or more sensitive areas of the medical station. In some implementations, more than one channel may extend from the first recess 116.

The raised covering component 110 has sidewalls 112a, 112b, 112c, and 112d extending from respective edges 114a, 114b, 114c, and 114d of the first top surface 118. The top surface 118 and the sidewalls 112a, 112b, 112c, and 112d form the protective structure (e.g., the raised covering component 110) in which the processing device of the medical station may be contained.

The planar support component 120 has a second top surface 122 that extends outward from a distal or bottom end 126 of the sidewall 112a of the raised covering component 110. A second recess 124 is formed within at least a portion of the second top surface 122. The second recess 124 is sized, shaped and configured to contain a liquid.

The first recess 116 may be defined by a first pair of opposing walls 116a and 116b, as shown in FIG. 2. The first pair of opposing walls 116a and 116b prevent spilled material from being directed to a front area of the raised covering component 110, where the display unit 150 may be positioned, and to a back area of the raised covering component 110, where access to the processing device may be provided. For example, on the sidewall 112c, a back plate 130 is provided and secured; see also FIGS. 1 and 4. The back plate 130 has various openings and connections to the processing device contained within the raised covering component 110. Thus, the opposing walls 116a and 116b prevent any spilled material from coming into contact with these sensitive areas. Moreover, the channel 117, which extends over the sidewall 112b from the first recess 116 between the opposing walls 116a and 116b, further diverts the spilled material from the display unit 150 and the back plate 130. In some implementations, the opposing walls 116a and 116b may be oriented and/or configured in a sloped orientation with respect to a bottom surface of the first recess 116. The slope can be oriented to direct fluid to any location such as to a rearward and/or frontward direction. Other structures can be included to direct fluid away from the station. For example, a lip can be included to direct fluid away from the front or rear surface so that fluid spills toward the floor away from the station.

Consistent with implementations of the current subject matter, two channels may be formed in the top surface 118 and extend from the first recess 116 and along the sidewalls 112b and 112d.

The second recess 124 of the planar support component 120 may be defined by a first pair of opposing walls and a second pair of opposing walls, the second pair of opposing walls oriented perpendicular to the first pair of opposing walls. One or more of the first pair of opposing walls and the second pair of opposing walls may be in a sloped orientation with respect to a bottom surface of the second recess 124. The second recess 124, formed in the second top surface 122, may serve to collect and contain spilled material in the front portion of the upper module 100.

According to some aspects of the current subject matter, the first recess 116 may have a concave configuration in which a middle portion or near middle portion of the first recess 116 is lower than surrounding portions. Similarly, the second recess 124 may have a concave configuration in which a middle portion or near middle portion of the second recess 124 is lower than surrounding portions.

In some implementations, the first top surface 118 and/or the second top surface 122 may have respective grated plates that fit within at least a portion of the respective recesses, the first recess 116 and the second recess 124. The grated plates may align with the first top surface 118 and/or the second top surface 122, and may have holes or openings through which a material that is spilled passes for collection and/or diversion.

As shown in FIG. 3, the first top surface 118 and the second top surface 122 are aligned in a side by side configuration, while at different heights from one another by virtue of the side walls 112a, 112b, 112c, and 112d that form the sides of the raised covering component 110. As seen in FIG. 3, and also in FIG. 1, the first top surface 118 includes a substantially flat area adjacent at least a portion of the first recess 116. The second top surface 122 includes a substantially flat area adjacent at least a portion of the second recess 124.

The first top surface 118, the first recess 116, the second top surface 122, and the second recess 124 may be formed from a hydrophobic material. The hydrophobic material may include a plastic or resin that repels liquid. In some implementations, portions of the surfaces or recesses may be formed from or coated with a hydrophobic material. Using hydrophobic material, either to form or coat a component, liquid can be diverted along a particular path and away from sensitive locations. Examples of hydrophobic materials include waxes, oils, alkyls, fluoroalkyls, prefluoroalkyls, or combinations or variants thereof. U.S. Pat. No. 9,096,786 describes spill-proof or spill-resistant surfaces through the use of hydrophobic or oleophobic and is incorporated herein by reference in its entirety.

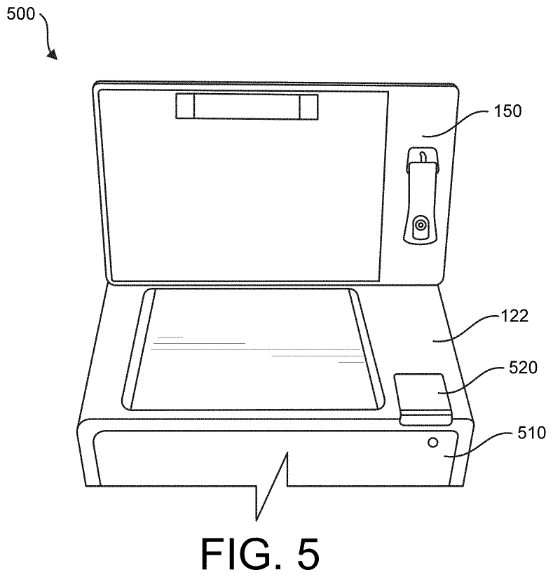

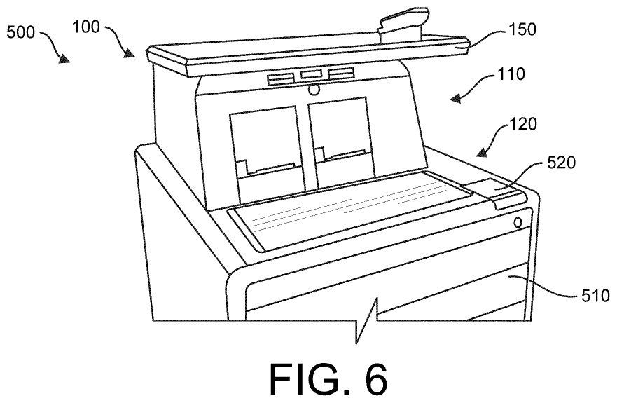

FIG. 5 and FIG. 6 depict aspects of a medical station 500 consistent with implementations of the current subject matter. The medical station 500 includes the upper module 100 having the raised covering component 110 and the planar support component 120 and a base 510. In some implementations, the base 510 and the upper module 100 are a single molded component. In some implementations, the upper module 100 is s releasably connectable to at least one portion of the base 510. The upper module 100 may form a hinged connection with at least one portion of the base 510. The first top surface 118 and the second top surface 122 of the upper module 100 are substantially perpendicular to base sidewalls of the base 510.

With reference still to FIG. 5 and FIG. 6, an electronic component 520 is positioned on or adjacent the second top surface 122 at a location accessible by a user. The electronic component 520 may be, for example, a card reader. Any portion of the second top surface 120 or the second recess 124 may be oriented to direct fluid to flow away from the electronic component 520 such as to avoid liquid damage to the electronic component.

Although the disclosure, including the figures, described herein may describe and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A medical station, comprising:
a base; and
an upper module formed of a structure positioned atop the base, the upper module comprising a raised covering component and a planar support component,
the raised covering component comprising:
a first top surface;
a first recess formed within at least a portion of the first top surface;
at least one channel extending from the first recess; and
a plurality of sidewalls extending from respective edges of the first top surface;
the planar support component comprising:
a second top surface extending from a distal end of a first sidewall of the plurality of sidewalls of the raised covering component; and
a second recess formed within at least a portion of the second top surface.

2. The medical station of claim 1, wherein the first top surface and the second top surface are aligned in a side by side configuration at different heights from one another.

3. The medical station of claim 1, wherein the base and the upper module comprise a single molded component.

4. The medical station of claim 1, wherein the upper module is releasably connectable to at least one portion of the base.

5. The medical station of claim 1, wherein the upper module forms a hinged connection with at least one portion of the base.

6. The medical station of claim 1, wherein the first top surface and the second top surface are substantially perpendicular to base sidewalls of the base.

7. The medical station of claim 1, wherein the first recess is defined by at least a first pair of opposing walls.

8. The medical station of claim 7, wherein the first pair of opposing walls are in a sloped orientation with respect to a bottom surface of the first recess.

9. The medical station of claim 7, wherein the at least one channel extends between the first pair of two opposing walls and along one of the plurality of sidewalls.

10. The medical station of claim 1, wherein the first top surface comprises two channels, a first channel extending from the first recess and along a first of the plurality of sidewalls, a second channel extending from the first recess and along a second of the plurality of sidewalls, the first of the plurality of sidewalls opposite the second of the plurality of sidewalls.

11. The medical station of claim 1, wherein the second recess is defined by a first pair of opposing walls and a second pair of opposing walls, the second pair of opposing walls oriented perpendicular to the first pair of opposing walls.

12. The medical station of claim 11, wherein one or more of the first pair of opposing walls and the second pair of opposing walls are in a sloped orientation with respect to a bottom surface of the second recess.

13. The medical station of claim 1, wherein the first recess comprises a concave configuration in which a middle portion of the first recess is lower than surrounding portions.

14. The medical station of claim 1, wherein the second recess comprises a concave configuration in which a middle portion of the second recess is lower than surrounding portions.

15. The medical station of claim 1, wherein the first top surface further comprises a grated plate fitted within at least a portion of the first recess.

16. The medical station of claim 1, wherein the second top surface further comprises a grated plate fitted within at least a portion of the second recess.

17. The medical station of claim 1, wherein the first top surface comprises a substantially flat area adjacent at least a portion of the first recess.

18. The medical station of claim 1, wherein the second top surface comprises a substantially flat area adjacent at least a portion of the second recess.

19. The medical station of claim 1, further comprising an attachment component affixed to the upper module, the attachment component configured to securely hold a display unit.

20. The medical station of claim 1, wherein the first recess and/or the second recess comprises a hydrophobic material.

21. The medical station of claim 1, wherein the medical station is part of a modular system.

22. The medical station of claim 1, wherein the medical station is a standalone module.

23. The medical station of claim 1, wherein the raised covering component is configured to cover and protect a processing device.

24. The medical station of claim 23, further comprising a back plate attached to one of the plurality of sidewalls, the back plate providing access to the processing device.

25. The medical station of claim 23, wherein the at least one channel diverts a material contained in the first recess from the back plate.

26. The medical station of claim 1, further comprising an electronic component on or adjacent the second top surface, wherein the second top surface or second recess is oriented to direct fluid to flow away from the electronic component such as to avoid liquid damage to the electronic component.
